(19) United States
(12) Patent Application Publication
(10) Pub. No.: US 2005/0043605 A1
(43) Pub. Date: Feb. 24, 2005

(54) System and method for measuring an electrocardiogram and communicating with an implanted device

(76) Inventors: Jeffrey A. Hall, Birmingham, AL (US); Neil Kay, Birmingham, AL (US)

Correspondence Address: MERCHANT & GOULD PC, P.O. BOX 2903, MINNEAPOLIS, MN, 55402-0903, US

(21) Appl. No.: 10/635,167

(22) Filed: Aug. 6, 2003

(51) Int. Cl. A61B 5/04

(52) U.S. Cl. 600/382; 600/509

(57) ABSTRACT

Systems and methods for measuring an electrocardiogram and communicating with an implanted device of an patient. The system may include a support structure including a support surface, as well as a communication system coupled to the support structure. The communication system may include a coil positioned to communicate with the implanted device when the patient is positioned on the support surface of the support structure. The communication system may be moveable with respect to the support structure to optimize communication with the implanted device. The system may also include a mat including a first surface and an electrode associated with the mat. The electrode may be positioned to make contact with the patient to allow for the measuring of the electrocardiogram alone or in conjunction with testing functionality of the implanted device. The mat may be positioned on the support structure.

### TECHNICAL FIELD

The present invention is directed to systems and methods to obtain and/or record electrocardiograms and to communicate with cardiac rhythm management devices.

### BACKGROUND

The heart is a muscular organ comprising multiple chambers that operate in concert to circulate blood throughout the body's circulatory system. The heart includes right and left atria and right and left ventricles. Oxygen-depleted blood returning to the heart from the body collects in the right atrium. When the right atrium fills, the oxygen-depleted blood passes into the right ventricle where it can be pumped to the lungs via the pulmonary arteries.

Within the lungs, waste products such as carbon dioxide are removed from the blood and expelled from the body, and oxygen is transferred to the blood. Oxygen-rich blood returning to the heart from the lungs via the pulmonary veins collects in the left atrium. The circuit between the right atrium and ventricle, the lungs, and the left atrium is generally referred to as the pulmonary circulation. After the left atrium fills, the oxygen-rich blood passes into the left ventricle, where it can be pumped throughout the entire body. In so doing, the heart is able to supply oxygen to the body and facilitate the removal of waste products from the body.

To circulate blood throughout the body's circulatory system, as described above, a beating heart performs a cardiac cycle that includes a systolic phase and a diastolic phase. During the systolic phase, or systole, the ventricular muscle cells of the right and left ventricles contract to pump blood through the pulmonary circulation and throughout the body, respectively. Conversely, during the diastolic phase, or diastole, the ventricular muscle cells of the right and left ventricles relax, during which the right and left atria contract to force blood into the right and left ventricles, respectively. Typically, the cardiac cycle occurs at a frequency between 60 and 100 cycles per minute and can vary depending on physical exertion and/or emotional stimuli, such as pain or anger.

The contractions of the muscular walls of each chamber of the heart are controlled by a complex conduction system that propagates electrical signals to the heart muscle tissue to effectuate the atrial and ventricular contractions necessary to circulate the blood. The complex conduction system includes an atrial node (the sinoatrial node) and a ventricular node (the atrioventricular node). The sinoatrial node initiates an electrical impulse that spreads through the muscle tissues of the right and left atria and the atrioventricular node. As a result, the right and left atria contract to pump blood into the right and left ventricles, as discussed above.

At the atrioventricular node, the electrical signal is momentarily delayed before propagating through the right and left ventricles. Within the right and left ventricles, the conduction system includes right and left bundle branches that extend from the atrioventricular node via the Bundle of His. The electrical impulse spreads through the muscle tissues of the right and left ventricles via the right and left bundle branches, respectively. As a result, the right and left ventricles contract to pump blood throughout the body, as discussed above.

An electrocardiogram (“ECG”) is a measurement or representation of the heart's electrical activity at a surface of a patient. An ECG is typically broken down into its significant components, such as P-waveform representing atrial depolarization, a QRS complex representing ventricular depolarization, and a T-waveform represents ventricular repolarization. An electrocardiograph, as is known in the art, may be used to obtain and/or record an ECG. Typically, an electrocardiograph, such as that disclosed in U.S. Pat. No. 4,793,361 to Cardiac Pacemakers, Inc., incorporated herein in its entirety, requires various electrodes to be attached to various surfaces of a patient's body (e.g., a patient's right and left arms as well as left leg) to obtain and/or record the ECG.

An ECG may be used by a caregiver to diagnose various heart abnormalities. If a person is diagnosed with a heart abnormality such as, for example and without limitation, bradycardia, tachycardia, and/or congestive heart failure, a cardiac rhythm management (“CRM”) device may be implanted in the patient to manage the abnormality. A CRM device may provide therapy for a patient and may communicate externally with a programmer to allow data to be exchanged between the CRM device and the programmer. Typically, communication between a CRM device and a programmer is initiated by placing a wand near a surface of a patient adjacent the CRM device. The ECG is usually used concurrently with the communication between the programmer and CRM device, such as a pacemaker, to evaluate the performance of the intra-cardiac electrical leads and troubleshoot device problems. For example, see U.S. Pat. No. 6,353,761 to Cardiac Pacemakers, Inc., incorporated herein in its entirety.

It is desirable to provide a system that will allow for efficient collection and/or recording of an ECG. It is also desirable to provide a system that will allow for efficient communication between a CRM device and an external programmer.

### SUMMARY

The present invention is directed to systems and methods to obtain and/or record electrocardiograms and to communicate with cardiac rhythm management devices.

In one aspect, the invention relates to an apparatus for obtaining and/or recording an electrocardiogram of a patient, the apparatus including a mat having a first surface, upper and lower portions, and an electrode associated with the mat. The electrode is positioned so as to make contact with the patient to allow for obtaining and/or recording of the electrocardiogram.

In another aspect, the invention relates to an apparatus for obtaining and/or recording an electrocardiogram of a patient, the apparatus including a mat having a first surface, first and second ends, and right and left sides, and a plurality of electrode buttons associated with the mat. The plurality of electrode buttons may be positioned to make contact with anatomical features of the patient to allow for measuring of the electrocardiogram.

In another aspect, the invention relates to an apparatus for communicating with an implanted device in an patient, the apparatus including a support structure having a support surface, and a system coupled to the support structure, the system including a coil, commonly known and used in the art for communicating with an implanted device, positioned to communicate with the patient's implanted device when the patient is positioned on the support surface of the support structure.

In another aspect, the invention relates to a system for obtaining and/or recording an electrocardiogram and communicating with an implanted device of a patient, the system including a support structure having a support surface and a communication system coupled to the support structure. The communication system includes a coil positioned to communicate with the implanted device when the patient is positioned on the support surface of the support structure. The system may also include a mat positioned on the support structure and including a first surface, first and second ends, right and left sides, and a plurality of electrode buttons associated with the mat, the plurality of electrode buttons being positioned to make contact with the patient to allow for the measuring of the electrocardiogram.

In another aspect, the invention relates to a method of obtaining and/or recording an electrocardiogram of a patient, which includes providing an electrode and a mat having a first surface, positioning the electrode on the mat to align with a anatomical feature of the patient, and obtaining and/or recording the electrocardiogram of the patient with the electrode.

In yet another aspect, the invention relates to a method of communicating with an implanted device in a patient that includes the step of providing a support structure having a support surface and a system coupled to the support surface that includes a coil. The method also includes the step of positioning the coil relative to the patient to communicate with the implanted device when the patient is positioned on the support surface of the support structure.

The above summary of the present invention is not intended to describe each disclosed embodiment or every implementation of the present invention. The figures and the detailed description which follow more particularly illustrate these embodiments.

### DESCRIPTION OF THE DRAWINGS

FIG. 1 is a top plan view of an example embodiment of an electrocardiographic mat made in accordance with the present invention.

FIG. 2 is a side plan view of the example electrocardiographic mat shown in FIG. 1 with a patient positioned on the mat.

FIG. 3 is a top plan view of another example embodiment of an electrocardiographic mat made in accordance with the present invention

FIG. 4 is a side view of a portion of the example electrocardiographic mat illustrated in FIG. 3 showing an example embodiment of an electrode-button made in accordance with the present invention.

FIG. 5 is a side view of a portion of the example electrocardiographic mat illustrated in FIG. 3 showing, in exploded form, an example embodiment of another electrode button including an example gel electrolyte cap.

FIG. 6 is a top plan view of the example electrocardiographic mat shown in FIG. 1 with additional electrode buttons.

FIG. 7 is a top plan view of another example of an electrocardiographic mat having electroconductive strips in accordance with the present invention.

FIG. 8 is a side plan view of an example embodiment of an apparatus for communicating with an implanted device of a patient.

FIG. 9 is a side cross-sectional view of the example apparatus shown in FIG. 8.

FIG. 10 is a side plan view of the example electrocardiographic mat of FIG. 1 placed on the example apparatus of FIG. 8.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Various embodiments of the present invention will be described in detail with reference to the drawings, wherein like reference numerals represent like parts and assemblies throughout the several views. Reference to various embodiments does not limit the scope of the present invention, which is limited only by the scope of the claims attached hereto.

The invention relates generally to a system for obtaining and/or recording an electrocardiogram and communicating with an implanted device of a patient. The system may include a support structure having a support surface and a communication system coupled to the support structure. The communication system may include a coil positioned to communicate with the implanted device when the patient is positioned on the support surface of the support structure. The system may also include a mat positioned on the support structure that includes a first surface, first and second ends, right and left sides, and a plurality of electrode buttons associated with the mat. The plurality of electrode buttons may be positioned on the mat to make contact with the patient to allow for measuring of the electrocardiogram.

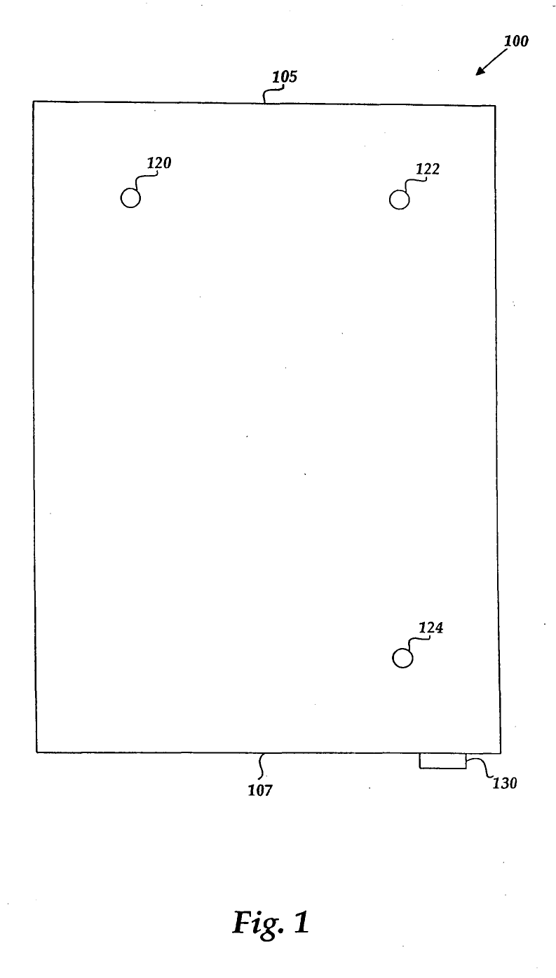

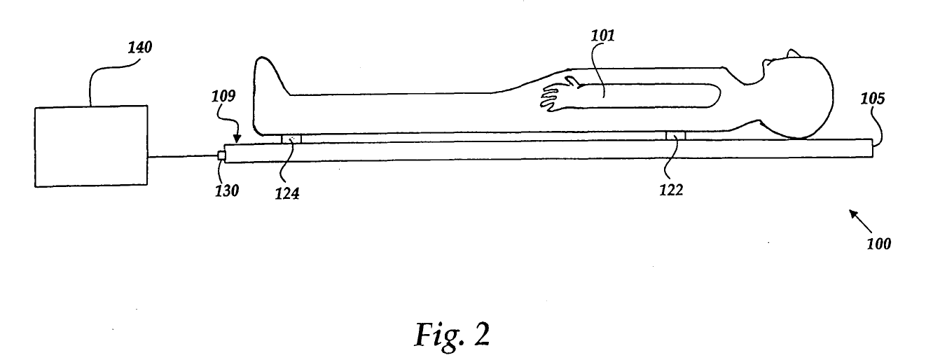

Referring now to FIGS. 1 and 2, an example embodiment of an electrocardiographic mat 100 is shown sized to accommodate at least a portion of a body of a patient 101. The mat 100 generally includes a first end 105, a second end 107, and a surface 109.

Associated with the surface 109 of the mat 100 are three electrodes buttons 120, 122, and 124. The three electrode buttons 120, 122, and 124 are electrically connected (using, for example, wiring running through the mat 100 (not shown)) to an output 130 that is configured to be connected to an electrocardiograph 140. The electrode buttons 120, 122, and 124 are positioned to make electrical contact with various portions of a patient's body when the patient is positioned on the mat 100 so that the patient's head extends towards the first end 1 OS and the patient's legs extend towards the second end 107. For example, the electrode button 120 is positioned to contact the patient's right arm, the electrode button 122 is positioned to contact the patient's left arm, and the electrode button 124 is positioned to contact the patient's left leg.

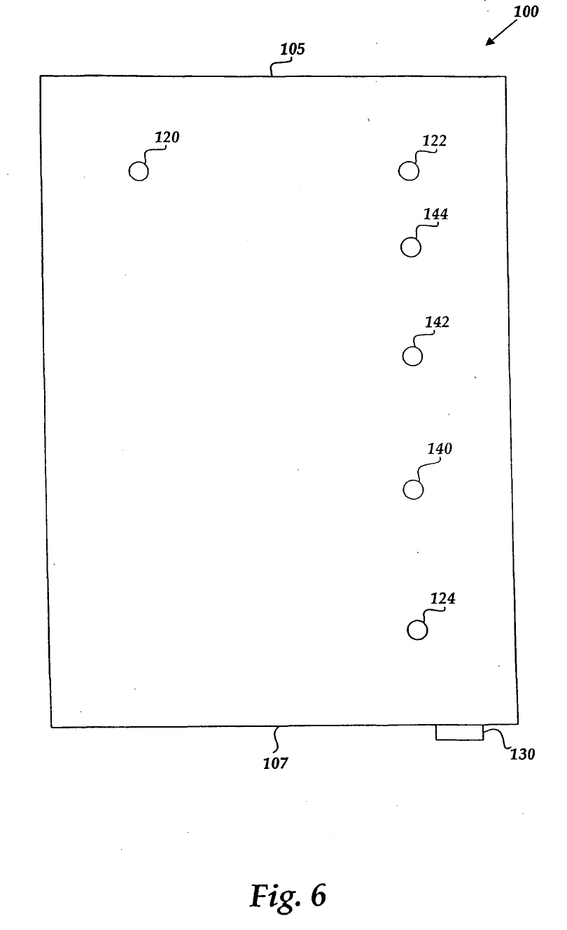

The mat 100 may also include additional electrode buttons positioned on the mat to make electrical contact with other portions of a patient's body. FIG. 6 illustrates electrode buttons 140, 142 and 144 that are positioned on the mat 100 to contact the patient's upper left leg, left lumbar, and left posterior thorax, respectively. The mat 100 may include one or all of the electrode buttons 120, 122, 124, 140, 142 or 144 in various embodiments of the present invention.

In the configuration of FIGS. 1 and 2, the mat 100 allows for the electrode buttons 120, 122 and 124 to make electrical contact with the surface of the patient 101 and to obtain and/or record the electrical activity of the patient's heart. By coupling the output 130 of the mat 100 to the electrocardiograph 140 (either hardwired or wirelessly using, for example, RF or IR technology), the patient's electrocardiogram (“ECG”) can be obtained and/or recorded.

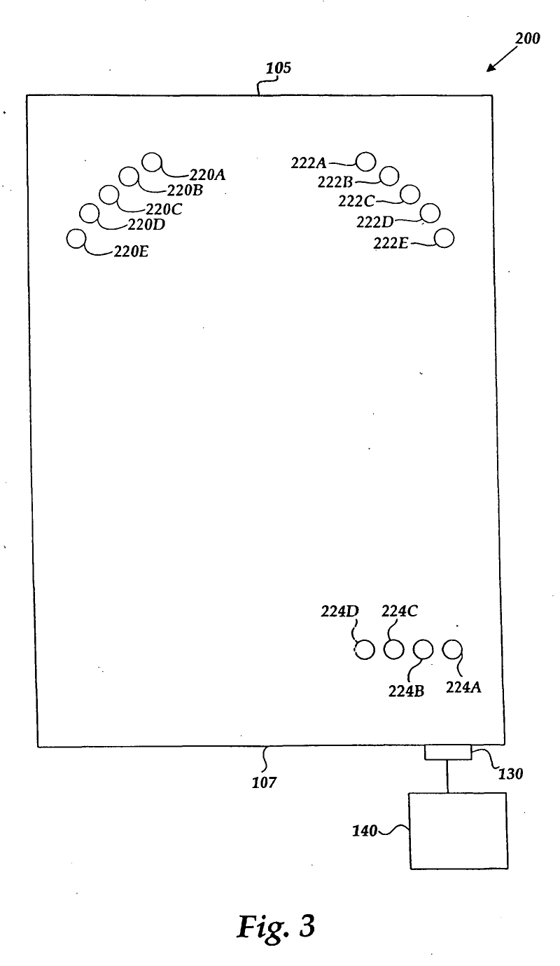

Referring now to FIG. 3, another example embodiment of an electrocardiographic mat 200 is shown. The mat 200 is similar to the mat 100, except that the mat 200 includes a plurality of electrode buttons 220A-220E, 222A-222E, and 224A-224D, positioned to make contact with various portions of a patient's anatomy. For example, electrode buttons 222A, 222B, 222C, 222D, and 222E are positioned so that optimal contact can be made between one or more of the buttons 222A-222E and a patient's left arm and shoulder. Any of the patient electrode buttons of the series of electrode buttons 220A-220E, 222A-222E, and 224A-224D may be mounted separately to mat 100 and wired individually. In other embodiments, each series of electrode buttons 220A-220E, 222A-222E, and 224A-224D may be mounted separately to a wire or other continuous conductive material.

In one example embodiment, each of the electrode buttons 222A-222E are exposed and one or more of the electrode buttons 222A-222E are automatically selected, for example, by the electrocardiograph 140 based on the optimum electrogram signal received from the electrodes, to provide optimal obtaining and/or recording of the electrical activity of the patient's heart. For example, the electrocardiograph 140 may use parameters or selection criteria such as slope, amplitude, contact impedance, noise, and electrode geometry to select the optimal electrode buttons for obtaining and/or recording of the ECG. One example of testing which electrode has achieved proper contact with the patient for optimal obtaining and/or recording of information is to send minute electrical pulses to each electrode button on a periodic basis and obtain and/or record the impedance of that electrode to assess the quality of contact with the patient.

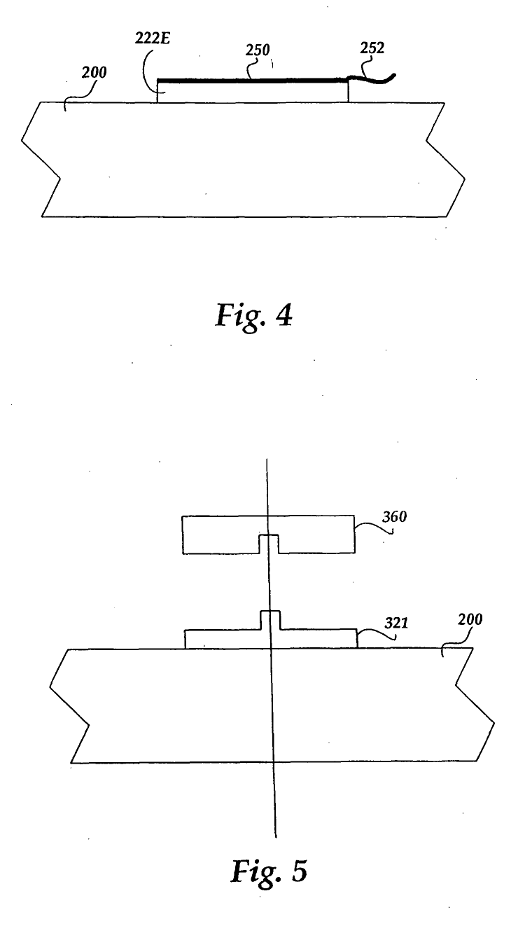

Alternatively, as shown in FIG. 4, each electrode button, such as electrode button 222E, may include a cover 250 made of, for example, paper or plastic. The cover 250 may include a tab 252 extending from the cover 250 to allow for ease in grasping and removing the cover 250. The cover 250 may be removed from one or more of the electrode buttons to expose the electrode buttons to take electrocardiographic measurements. For example, depending on the size of a patient, it may be advantageous to expose the electrode button 222A closer to the first end 105 if the patient is tall or the electrode button 222E closer to the second end 107 if the patient is short.

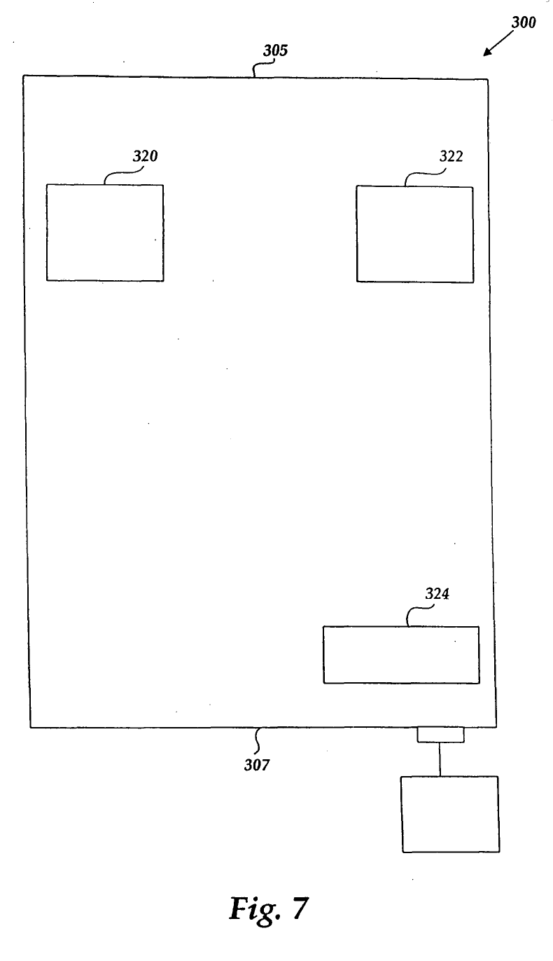

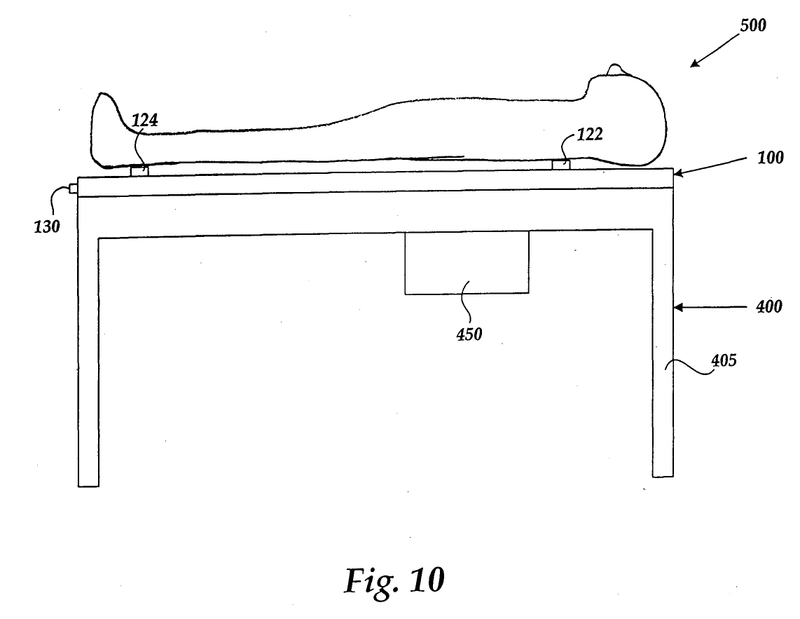

Referring now to FIG. 7, another example embodiment of an electrocardiographic mat 300 is shown. The mat 300 is similar to the mat 200, except that the mat 300 includes strips of electroconductive material 320, 322 and 324 positioned to make contact with various portions of a patient's anatomy. The size and shape of strips 320, 322 and 324 may be configured to account for a variety of different anatomical sizes. Furthermore, the strips 320, 322 and 324 may be sized and shaped differently from each other. For example, strips 320 and 322 may be longer (length measured between first and second ends 305 and 307 of the mat 300) than strip 324, and strip 324 may be wider than strips 320 and 322 (see FIG. 10).

In a yet further example (not shown), the electrodes are positioned on a pivoting arm that is movable in at least two directions to optimize proper positioning of the electrodes with anatomical features of a give patient. For example, separate pivoting arms with at least one electrode mounted to each arm may be mounted to the mat in positions that correspond to the upper left and right sides and lower leg of the patient (for example, the position of electrodes 120, 122, 124 shown in FIG. 1). The pivoting arms may be mounted above or below the contact surface of the mat so long as the electrode mounted to the pivot arm is capable of being exposed for contact by the patient.

The electrocardiographic mats 100, 200 and 300 may be made of a variety of materials. For example, the mats may be composed of a fibrous material such as paper or be made of a polymer (e.g., plastic) or a foam material. The mats may be disposable or reusable. For example, the mats may be made of a disposable paper sheet that is discarded after each use. Alternatively, the mats may be made of a smooth plastic material that is reusable and allows for easy cleaning. A hybrid mat with a portion that is disposable and a portion that is reusable is also possible.

The mat may also have multiple layers (not shown). For example, the mat may be composed of an upper layer that provides a surface for direct contact by the patient and a lower layer that each act as insulators, and a middle layer comprising a conductive material. In such a configuration, the conductive material may be divided into separate sections with each section being electrically connected to the electrocardiograph. The upper layer may be configured to facilitate an electrical connection between an electrode and the conductive material, such as, for example, by piercing through the upper layer. Thus, an electrode could be positioned at a variety of places on the mat for optimum alignment with desired anatomical features of any given patient.

In another embodiment, a mat having multiple layers may be inflatable to promote contact between the electrode associated with the mat and a patient in contact with the mat. For example, the mat may have electrodes mounted to it as shown in FIG. 1, and the top surface of the mat is flexible so as to match the contours of a patient's body as the mat is inflated.

In a yet further embodiment, the mat may have a contact surface that is contoured to match anatomical features of a patient. For example, the mat may have recessed areas in the contact surface of the mat that align with the shoulders and buttocks of the patient. In such an embodiment, the electrodes may be positioned in the recessed areas for contact with predetermined anatomical features of the patient.

The electrodes may be made of any material typically used for electrodes, such as, for example, stainless steel or conductive gel material. The electrode buttons or strips may be disposed within the mat 100, coupled to the surface 109, or otherwise associated with the mat. As noted above, the electrodes may be exposed or covered (see the example button of FIG. 4), or may be flush with an upper surface of the mat or raised relative to an upper surface of the mat. Alternatively, as illustrated in FIG. 5, each electrode may include a base 321 disposed in the mat 200 and a detachable gel electrolyte cap 360. The cap 360 can be coupled to the base 321 and can enhance the electrical connection created between the electrode button and the surface of the patient. The caps 360 can be detached and replaced as needed.

Other configurations for the electrodes are also possible. For example, the cap or base may be available in different sizes or shapes to achieve improved contact with the patient. The electrodes may be rectangular (such as strips 220, 221 and 222), circular (such as electrode buttons 120, 121 and 122), or a similar shape, and have varying sizes depending on the requirements and purpose of a particular embodiment.

In yet further embodiments, some of the electrodes may be permanently mounted to the mat while other electrodes are temporarily mounted to the mat. For example, electrodes associated with the upper body of the patient may be embedded in the mat in a permanent state while an electrode associated with a leg of the patient is temporarily mounted to the mat in a desired position for a given patient.

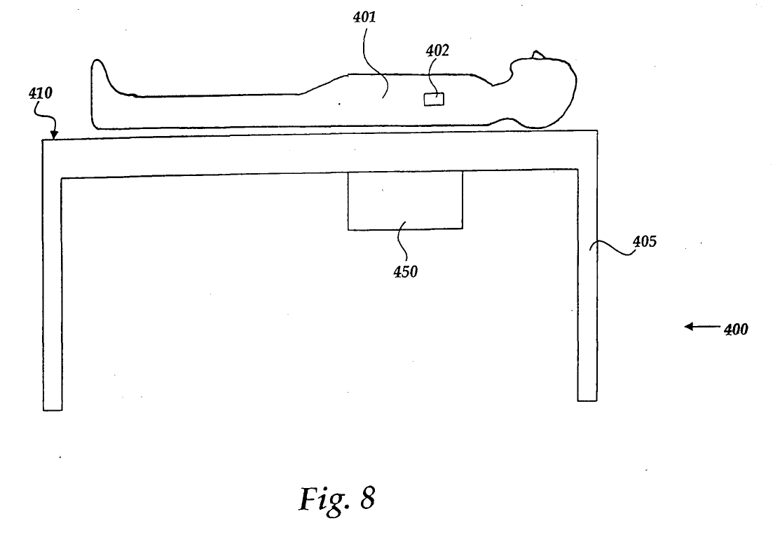

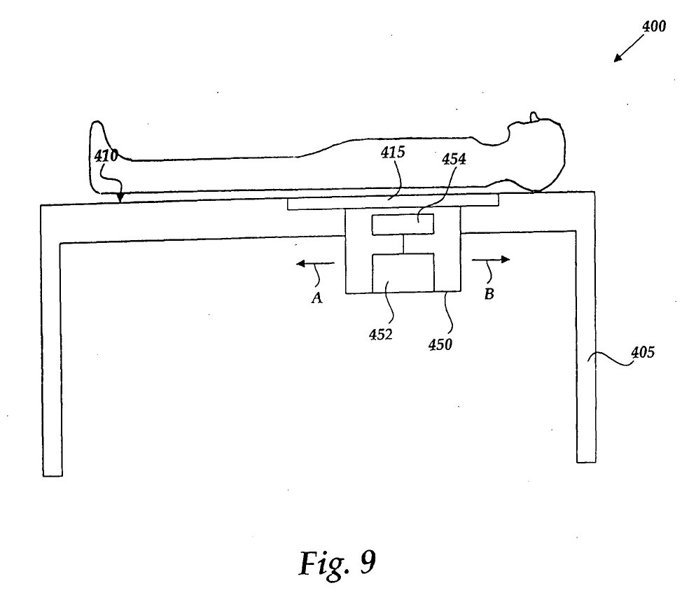

Referring now to FIGS. 8 and 9, an example embodiment of an apparatus 400 for communicating with an implanted device 402 (for example, a cardiac rhythm management device) in a patient 401 is shown. Generally, the apparatus 400 includes a support structure 405 with a support surface 410 and a system 450 coupled to the support structure. The system 450 includes a coil 454 to communicate with implanted device 402 and may also include a positioning device 452 to position coil 454. Coil 454 may be any coil commonly known in the art for use in communicating with a patient's implanted device. One example of a coil is an impedance coil that communicates with the patient's implanted device using impedance induced telemetry.

The system 450 is configured to communicate with the implanted device 402 when the patient is positioned on the support surface 410 of the support structure 405. The system 450 may, for example, download information collected by the implanted device 402. The system 450 may also, for example, upload information to the implanted device 402, such as software updates to modify therapy provided by the implanted device 402. The system 450 may be controlled by and/or communicate with other computing systems that may store and analyze information obtained and/or recorded from the implanted device 402 and provide additional information to be communicated to the implanted device 402.

The system 450 may be coupled to the support structure 405 in such a manner to allow the system 450 to be moved in directions A and B to optimize communication between the coil 454 and the implanted device 402. For example, the system 450 may be manually moveable by a caregiver in the directions A and B. A partially transparent portion 415 of the support surface 410 may assist the caregiver in positioning the coil 454 over the implant 402.

Alternatively, the system 450 may include the positioning device 452, which automatically moves the system 450 in the directions A and B to optimize communication between the coil 454 and the implanted device 402. For example, the positioning device 452 may move the system 450 in first the direction A and then the second direction B to identify a position at which communication is strongest between the system 450 and the implanted device 402. In other embodiments, the system 450 may be moveable in additional directions, such as directions generally transverse to the directions A and B to allow communication between the coil 454 and the implanted device 402 to be further optimized. Freedom of movement in any direction within a plane may also be provided, as well as allowing the system 450 to move in the vertical direction.

The implanted device 402 may be, for example, a cardiac rhythm management device that provided therapy and records information from the heart. In other embodiments, the implanted device 402 may include other implanted devices such as, for example, neurological devices, insulin pumps, and other implanted devices.

The apparatus 400 may be advantageous to allow for efficient, semi-automatic or automatic communication with an implanted device such as a cardiac rhythm management device. Further, if an automated system is used, communication with the implanted device can be optimized through automatic positioning of the coil in relation to the implant. The coil would be positioned relative to the implant in such an automated system to maximize the strength of the signal being transmitted between the coil and the implant.

Referring now to FIG. 10, an example embodiment of an apparatus 500 that includes the electrocardiographic mat 100 and the apparatus 400. The matt 100 of apparatus 500 includes at least electrode buttons 122 and 124 (see FIGS. 1 and 2) and output 130. The apparatus 400 of apparatus 500 includes the system 450 for communicating with an implanted device 402 (see FIG. 8). In this configuration, the apparatus 500 may facilitate the efficient obtaining and/or recording of a patient's ECG as well as provide communication between the patient's implanted device and the system 450. In this configuration, the obtaining and/or recording of the patient's ECG is used primarily to test functionality of the patient's implanted device. This type of testing typically requires readings from only two or three electrodes (“leads ”) on the mat, such as electrode buttons 122, 124 or any of the electrodes shown with respect to apparatus 200 and apparatus 300.

In other embodiments, the mat may be configured to obtain and/or record a six or twelve lead ECG alone or in conjunction with testing functionality of the patient's implanted device. Six and twelve lead ECG's typically require a specific pattern of electrode placement relative to certain anatomical features of the patient, such as, for example, obtaining and/or recording information from electrodes positioned in a pattern around the patient's heart. Since six and twelve lead ECG's are typically obtained and/or recorded from electrodes mounted to a patient's chest, positioning of the electrodes on the mat of the present invention for obtaining and/or recording information through the posterior of the patient would be modified in order to obtain and/or record proper ECG information.

A method of measuring an electrocardiogram of a patient may include providing an electrode and a mat having a first surface, positioning the electrode on the mat to align with a anatomical feature of the patient, and measuring the electrocardiogram of the patient with the electrode. A method of communicating with an implanted device in a patient may include the step of providing a support structure having a support surface and a system coupled to the support surface that includes a coil, and the step of positioning the coil relative to the patient to communicate with the implanted device when the patient is positioned on the support surface of the support structure.

While the invention has been particularly shown and described with reference to preferred embodiments thereof, it will be understood by those skilled in the art that various other changes in the form and details may be made therein without departing from the spirit and scope of the invention.

1. An apparatus for measuring an electrocardiogram of a patient, comprising:
a mat including a first surface, upper and lower portions, and right and left sides; and
an electrode associated with the mat, the electrode being positioned to contact the patient and obtain the patient's electrocardiogram.

2. The apparatus of claim 1, wherein the electrode is positioned to contact an upper body of the patient when the patient is in contact with the first surface of the mat.

3. The apparatus of claim 1, wherein the electrode is an electrode button.

4. The apparatus of claim 1, wherein the electrode is an electroconductive strip.

5. The apparatus of claim 1, wherein the electrode comprises a plurality of electrodes positioned on the mat to optimize contact between the electrodes and anatomical features of the patient.

6. The apparatus of claim 5, wherein the plurality of electrodes includes:
at least one electrode positioned generally on the right side of the upper portion of the mat corresponding to a right shoulder or right upper arm of the patient; and
at least one electrode positioned generally on the left side of the upper portion of the mat corresponding to a left shoulder or left upper arm of the patient; and
at least one electrode position generally in the left side of the lower portion of the mat corresponding to a lower lumbar, left buttock, or left leg of the patient

7. The apparatus of claim 1, wherein the electrode comprises a contact surface for contacting the patient that is raised relative to the first surface of the mat.

8. The apparatus of claim 1, wherein the electrode includes:
a base associated with the mat; and
a detachable gel electrolyte cap.

9. The apparatus of claim 1, wherein at least a portion of the mat is disposable after use.

10. The apparatus of claim 1, wherein the mat is made of a polymer.

11. The apparatus of claim 5, further comprising a device to automatically select which of the plurality of electrodes to use in obtaining the electrocardiogram based on selection criteria.

12. The apparatus of claim 11, wherein the selection criteria includes factors selected from the group consisting of but not limited to: slope, amplitude, contact impedance, noise, and geometry of the electrical signal from the electrodes.

13. The apparatus of claim 12 wherein the contact impedance is obtained by sending minute electrical pulses to the electrode to obtain the impedance of the electrodes to assess contact between the electrodes and the patient.

14. An apparatus for communicating with an implanted device in a patient, comprising:
a support structure including a support surface; and
a system coupled to the support structure, the system including a coil positioned to communicate with the implanted device when the patient is positioned on the support surface of the support structure.

15. The apparatus of claim 14, wherein the system is at least partially disposed within the support structure.

16. The apparatus of claim 14, wherein the system is configured to communicate with the implanted device through a posterior of the patient.

17. The apparatus of claim 14, wherein the coil of the system is moveable to position the coil so that communication between the system and the implanted device is optimized.

18. The apparatus of claim 17, further comprising a positioning device coupled to the coil to automatically position the coil to optimize communication.

19. The apparatus of claim 17, wherein the coil is manually moveable.

20. The apparatus of claim 14, wherein at least a portion of the support surface is semi-transparent to allow the coil to be visible through the support surface.

21. The apparatus of claim 20, wherein the semi-transparent portion includes plexi film.

22. A system for measuring an electrocardiogram and communicating with an implanted device of a patient, the system comprising:
a support structure including a support surface;
a communication system coupled to the support structure, the communication system including a coil positioned to communicate with the implanted device when the patient is positioned on the support surface of the support structure;
a mat positioned on the support structure and including a first surface, first and second ends, and right and left sides; and
an electrode associated with the mat, the electrode being positioned to make contact with the patient to allow for measuring of the electrocardiogram.

23. The system of claim 22, further comprising a plurality of electrodes positioned on the first surface of the mat to align with anatomical features of the patient.

24. The system of claim 22, wherein the coil of the system is moveable to position the coil so that communication between the system and the implanted device is optimized.

25. An apparatus for measuring an electrocardiogram of a patient, comprising
a mat including a first surface, first and second ends, upper and lower portions, and right and left sides; and
a plurality of electrode buttons associated with the mat, the plurality of electrode buttons positioned to make contact with anatomical features of the patient to allow for the measuring of the electrocardiogram.

26. The apparatus of claim 25, wherein the plurality of electrode buttons includes at least a right electrode button positioned generally on the right side and upper portion of the mat to optimize contact with a right shoulder of the patient, and a left electrode button positioned generally on a left side and upper portion of the mat to optimize contact with a left shoulder of the patient.

27. The apparatus of claim 25, wherein the plurality of electrode buttons further includes a left leg electrode button positioned generally on the left side and lower portion of the mat to optimize contact between the left leg electrode button and a left leg of the patient.

28. The apparatus of claim 25, wherein the plurality of electrode buttons includes:
a right series of electrode buttons positioned generally on the right side and upper portion of the mat; and
a left series of electrode buttons positioned generally on the left side and upper portion of the mat; and
a left lower series of electrode buttons positioned generally on the left side and lower portion of the mat corresponding to the left lower body of the patient.

29. A method of measuring an electrocardiogram of a patient, comprising:
providing a mat including a first surface, first and second ends, right and left sides, and electrodes;
positioning the electrode on the mat to align with predetermined anatomical features of the patient; and
measuring the electrocardiogram using signals from the electrodes.

30. The method of claim 29, further comprising a plurality of electrodes aligned with each predetermined anatomical feature of the patient, wherein an electrode providing an optimal signal is automatically selected for each predetermined anatomical feature.

31. A method of communicating with an implanted device in a patient, comprising:
providing a support structure comprising a support surface, and a system coupled to the support surface, wherein the system includes a coil;
positioning the coil relative to the patient to communicate with the implanted device when the patient is positioned on the support surface of the support structure.

32. The apparatus of claim 31, wherein the coil of the system is moveable to position the coil so that communication between the system and the implanted device is optimized.

33. An apparatus for measuring an electrocardiogram of a patient, comprising:
an electrode configured to contact the patient and obtain and/or record the patient's electrocardiogram; and
means for mounting and positioning the electrode so as to facilitate contact between the electrode and predetermined anatomical features of the patient.
